(19) United States
(12) Patent Application Publication
(10) Pub. No.: US 2015/0075238 A1
(43) Pub. Date: Mar. 19, 2015

(54) PROCESS FOR PREPARING ACTIVATED VEGETABLE COMPLEXES AND CARBONATED, DOPED OR SUPERDOPED VEGETABLE/ORGANIC MATTERS COMPLEXES, AND APPLICATIONS OF SAME, ESPECIALLY FOR METHANATION OR PRODUCTION OF BIOGAS

(71) Applicant: Marcel Léon MEZY, Bozouls (FR)

(72) Inventor: Marcel Leon Mezy, Bozouls (FR)

(21) Appl. No.: 14/390,195

(22) PCT Filed: Mar. 28, 2013
(86) PCT No.: PCT/IB2013/000550

§ 371 Date: Oct. 2, 2014

(30) Foreign Application Priority Data
Apr. 5, 2012 (EP) 12 002 508.5

(51) Int. Cl. C05F 9/04; C05F 17/00; C12P 5/02

(52) U.S. Cl. CPC C05F 9/04; C12P 5/023; C05F 17/0027
USPC 71/9; 435/167; 585/16

(57) ABSTRACT

(EN)The invention relates to the preparation of doped plant complexes by a process of fermenting a compost CP in particular of straw and of horse manure fermented for 3-6 days, with coverage by a special carbonated plant complex CVC. A doped or overdoped plant complex is obtained that has a very high concentration in particular of humic acid nuclei, mycorrhizae, and fixed gases (nitrogen, carbon), having an extremely improved biological activity with an application in the improvement of methanization, up to 200-350%.

### TECHNICAL FIELD

The present invention relates to the technical field of organic and vegetable matters, biomasses and composts in general, of the preparation of complex composts, of diverse forms of presentation and of the applications thereof, especially for methanation.

According to the invention, there has been discovered:
a process of preparation,
from vegetable complexes (complexes of diverse plants CPL) and from a covering element such as straw P,
on the one hand a) from what we will call “vegetable complexes” CV here for simplicity and will explain that they are extremely activated,
on the other hand b) from what we will call “doped” or “superdoped vegetable complexes” here (R1 or R2) for simplicity.
These expressions relate to the attached figures and will be explained in detail hereinafter.

It will be seen that the present invention leads to doped or superdoped vegetable complexes, a complex that acts in very varied aerobic and anaerobic environments. In particular, it has the effect:
of accelerating the action of aerobic and anaerobic microorganisms by 1.1 to 10 times more and generally by 1.3 to 3 times more—especially in composting and methanation;
under anaerobic conditions:
of increasing the production of biogas and the proportion of methane in this biogas, which is equivalent to increasing the production of methane by 1.1 to 6 times more and generally by 1.3 to 3 times more and of reducing the proportion of CO₂ in the biogas by 10 to 90% and generally by 40 to 60%;
of reducing the emissions of H2S by 30 to 80% and generally by 40 to 60%;
of increasing the content of humic acids in the methanation digestate by 1.1 to 6 times more and generally by 1.3 to 3 times more and therefore of commensurately improving its fertilizing value;
under aerobic conditions:
of accelerating the fixation of the elements of the air, especially of increasing the fixation of CO₂, O₂ and N₂ by 1.1 to 10 times more and generally by 1.3 to 3 times more;
under aerobic and/or anaerobic conditions:
of accelerating the transformation of organic matters—especially the evolution, decomposition and synthesis of organic matters—by 1.1 to 10 times more and generally by 1.3 to 3 times more;
of reducing the gaseous and liquid releases/losses from organic and mineral matters during storage, evolution and spreading thereof, especially of nitrogen and carbon, by 10 to 90% and generally by 40 to 60%;
of accelerating the extraction and fixation of organic and mineral elements in the environment to which the invention relates, by 1.1 to 10 times more and generally by 1.3 to 3 times more, especially as an accelerator of the fixation of nitrogen and carbon by creating in particular “carbon and nitrogen sinks” useful in the fight against global warming and greenhouse gases;
of depolluting waters by reducing the COD by 10 to 90% and generally by 40 to 60%;
in the soils or the substrate (especially compost, culture medium, organic waste to be treated, farm effluents, manures, slurries, etc.) in which the invention is used under aerobic or anaerobic conditions:
of accelerating the production of humus and humic acids by 1.1 to 10 times more and generally by 1.3 to 3 times more, with the following consequences for the soil or the substrate in which the invention is used:
   an increase of the clay-humic complex, especially of its size and of its CEC (cation exchange capacity) by 1.1 to 10 times more and generally by 1.3 to 3 times more,
   an improvement of its structure, of its capacity to dry out in case of excess water, of its workability, of its ability to promote root development by 1.1 to 10 times more and generally by 1.3 to 3 times more,
   an improvement of its capacity to retain and save water, especially from irrigation and especially in a context of the fight against drought by 1.1 to 10 times more and generally by 1.3 to 3 times more,
   an increase of its resistance to erosion by 1.1 to 10 times more and generally by 1.3 to 3 times more,
   an improvement of its fertility, especially of the quantity of nutritive elements available for the plants by 1.1 to 10 times more and generally by 1.3 to 3 times more and of the balance of elements in the soils;
of increasing root development by 1.1 to 10 times more and generally by 1.3 to 3 times more;
of increasing the development of rhizospheric microorganisms, especially of micorrhizae, by 1.1 to 10 times more and generally by 1.3 to 3 times more;
of increasing the restitution of nutritive elements to plants by 1.1 to 10 times more and generally by 1.3 to 3 times more;
of increasing the resistance of plants to diseases by 1.1 to 10 times more and generally by 1.3 to 3 times more, and therefore of reducing the use of pesticide products by 10 to 100% and generally by 50 to 100%;
of increasing the biodiversity of plants in meadows and in multi-species cultures by 1.1 to 10 times more and generally by 1.3 to 3 times more;
of reducing the weeds by 10 to 90% and generally by 40 to 60%;
of increasing the quality and/or the quantity of agricultural and food production by 1.1 to 10 times more and generally by 1.3 to 3 times more, and therefore of improving the qualitative and quantitative nutrition of human beings by 1.1 to 10 times more and generally by 1.3 to 3 times more, in the context of the rising global population;
of increasing the extraction of unexploited mineral elements from soils, subsoils and substrates in which the invention is used, by 1.1 to 10 times more and generally by 1.3 to 3 times more;
of increasing the fixation and absorption of heavy metals, of salts, especially nitrates, or of any other pollutant in a contaminated soil or substrate, by 1.1 to 10 times more and generally by 1.3 to 3 times more, which has the consequence of reducing the transfer of these pollutants to the plants growing therein by 10 to 90% and generally by 40 to 60%.

NB: The term “organic matters” is used in its broadest sense, meaning that it corresponds to any material containing carbon.

Without wishing to be bound by any theory, the Applicant suggests that the properties (the surprising and highly favorable character of which, especially for methanation, will be measured) are due to the combination of either novel or strongly “doped” or “activated” reactions between solid/liquid/gas interfaces and gas phases and/or the bacteria, fungi, spores and other microorganisms that are present (and with the gas phases of the ambient atmosphere) during the use of the original process of the invention, especially initial degradation of vegetable phases, synergy with effects of covering a mass by a hearth, fixation of essential gas phases such as nitrogen or CO₂, and extraordinarily increased corollary productions of known or novel substances such as nodules of humic acids and mycorrhizae by virtue of multiplied exchanges between phases and slow percolation by gas phases.

### PRIOR ART AND TECHNICAL PROBLEMS

Naturally composts have been known for a very long time. They are more or less advanced decomposition products of natural products (leaves, grasses, etc.) or they result from matters of animal origin (manure, etc.) or from mixtures of these two types.

The compositions vary according to the soil from which they originate, the region, the animals in question, etc.

They involve slow processes, which by the fact of their slow nature allow numerous elements, in particular carbon and nitrogen, which have would proved very useful, to be lost to the environment (especially the air and the waters).

The use of bacteria to treat residual waters of all types is also known.

Finally, it is known to use organic matters to produce methane gas (CH₄). Nevertheless, the yield from the activity of bacteria producing in particular methane gas in well known manner is very low, and the use of methane is therefore poorly efficient, therefore quasi “anecdotal”, even though it could become an important source of energy. Here again the invention achieves an improvement representing a quantum leap.

Even though these problems are well known and have been for a long time, the industry in question has not found any technical solution and has therefore been content to develop palliative measures that do not resolve the entirety of the problems, and are very far from doing so.

A brief mention will be made of the agricultural sector, where a highly effective ecological and economic treatment of all residual waters with regard to the general properties listed hereinabove, but especially the methane sector has evolved little if at all, and to date no research seems to be of such nature as to permit high efficiency and no intensive research is being conducted in this respect (even though the production of so-called “green” or “clean” energy has experienced a recent and very strong boom (wind power, photovoltaic panels, etc.), which certainly shows that the demand for such forms of energy is significant and not satisfied.

### GENERAL DESCRIPTION OF THE INVENTION

“The person skilled in the art” is in the present Application any specialist making a living from the land, such as breeders, farmers, foresters and analogous activities, and having normal knowledge of fermentations, composts, “biomass” and of methane production or “methanation”.

The technical problem posed is to produce methane-containing biogas or methane by a methanation process; with a very greatly improved yield of CH₄.

### DETAILED DESCRIPTION OF THE INVENTION AND TECHNICAL SOLUTION

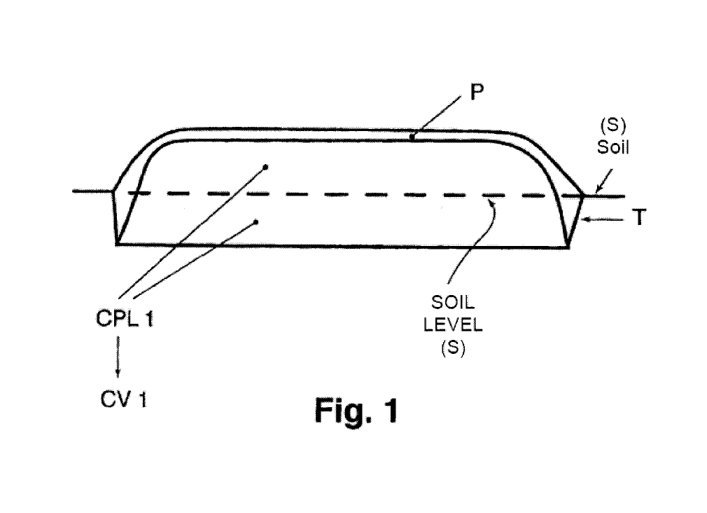

Reference will be made to the attached FIGS. 1 to 6.

The invention relates to a process for preparing doped or superdoped vegetable complexes CVD or CVSD said to be “with coverage of a compost by at least one vegetable complex”, characterized in that it comprises the following steps:

a) preparation of several, preferably 6 to 20, or preferably 8 to 15 activated vegetable complexes CV by using a layer of untreated straw to cover several spreadings of (6 to 20, or 8 to 15) “plant complexes”, each obtained from a plant or particular vegetable matter, and degradation of these products either outside the soil for 2 to 8 and preferably 3 to 6 months or half-buried in the soil for 18 to 24 months and preferably 2 years, then at the ends of these periods harvesting of these degradation products, which form a “vegetable complex CV”;

b) drying of the CV to a concentration of approximately 60-80% of dry matters;

c) mixing of the dried CV with a carbonate matter, especially a calcium carbonate, in proportions of 70 to 95% by weight of carbonate matter, thus forming a “carbonated vegetable complex” CVC;

d) in parallel, aerobic fermentation of a vegetable compost containing approximately 5-15% of organic matters for 3-6 days, which produces a “fermented or fermenting compost” CP;

e) preparation of a pile of this compost CP, which is covered:
by a layer of CVC
and preferably, in addition, by a top layer of activated coarse matters GA formed by the 1-mm screen rejects of the doped vegetable complex CVD obtained during production of a previous lot or “batch”;

f) harvesting of the upper part d2 of the compost CP situated underneath the interface with the layer of CVC, of the layer of CVC and, when it exists, of the layer of GA, when gaseous release is no longer observed at the top of the pile, which in its layer of GA yields the doped vegetable complex CVD (used under e) or, with the layer GA and transfer onto the 1-mm sieve, the superdoped vegetable complex CVSD, the two complexes being transferred onto the 1-mm sieve and the particles passing though the sieve being able to be used in pure or diluted form together with mineral or mineral/organic matters.

In more detailed manner, this process may be defined by the following steps:

Step A
Preparation of “activated vegetable complexes (CV)”, which may differ from one another as a function of the soils and analogous factors, or mixtures or associations of locally available and natural vegetable or mineral matters, neither polluting nor toxic.
As will be seen, these vegetable complexes CV are already highly “activated”.

Step A1

A certain number, between 6 and 20 types, preferably 10 to 15 types, depending on the local possibilities, of vegetable matters, to be referred to here as “plant complexes” or CPL, is harvested.

Each CPL will consist of a single plant or particular vegetable matter (or in the extremely predominant case of a single plant or particular vegetable matter, a slight contamination by other vegetable matters being able to be tolerated, because it is often inevitable during collection and sorting).

These plants or vegetable matters will preferably (but not limitatively) be chosen from among:
nettles,
oak leaves,
fungi, molds and spores present on the soil, developing underneath fallen leaves, for example, or in the soil, on roots, etc.
other plants or vegetable matters, depending on local availabilities.
Each plant or vegetable matter will be spread separately over a large area, for example one hectare, possibly half-buried in a trench T excavated in the soil (S), and in this way the nettles will therefore be spread over one area, the leaves over another, the fungi over a third, etc.

Step A2

1 First fraction of these plants or vegetable matters: FIG. 1

Each plant or vegetable matter CPL1 of this first fraction is spread over a large area, for example one hectare, half-buried in a trench T and half “outside the soil”.

Nettles and oak leaves and possibly other plants are particularly involved here.

This first option has been represented in FIG. 1, where a CPL1 (plant complex 1, for example oak leaves OR separately nettles, etc.) is half spread in a trench T, the other half (approximately) being outside the soil. The soil level is represented by (S).

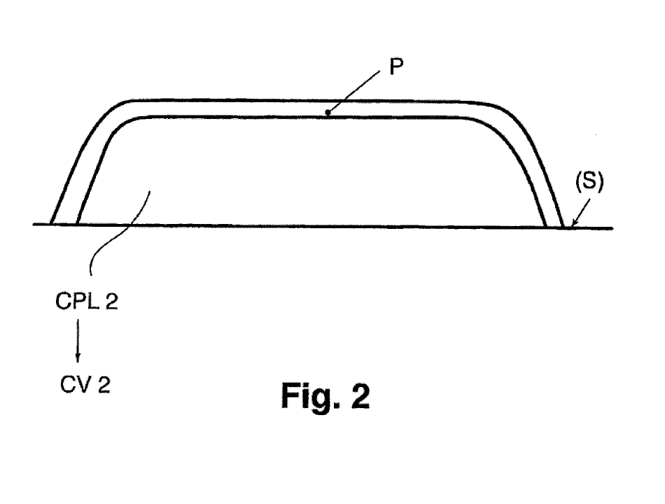

2 Second fraction of these plants or vegetable matters: FIG. 2

Each plant or vegetable matter CPL2 of this second fraction is spread over a large area, for example one hectare, totally “outside the soil”.

Leaves, fungi and other plants are particularly involved.

3 Each of these “spreadings” of plants or vegetable matters is covered by a thickness of UNTREATED straw P, especially a wheat or spelt straw.

4 Each “spreading” preferably has a thickness on the order of 80 cm to 1 m, and the thickness of the layer of straw P is on the order of 20 to 40 cm.

Incidentally, it is noted that, in the course of months, the “pile” will settle because of the degradation of the vegetable matters.

At the time of the “harvest”, the thickness of the plants will be reduced to around 15 to 20-30 cm.

Step A3

5 All of the spreadings are left to degrade for, by way of effective example, 18 to 24 months, preferably 2 years, for the half-buried spreadings of the half-buried “first fraction” and for 2 to 8 months, preferably 3 to 6 months, for the spreadings totally outside the soil, of the “second fraction”.

After the harvest (see hereinafter), there is surprisingly noted a very extensive degradation of the plants and vegetable matters and a very surprising growth of the population of fungi, spores, etc., and it seems that this surprising effect is due to the cover of straw, the degradation of which synergically influences the degradation of the plants and vegetable matters, with an additional covering effect which probably greatly slows the losses of matters and favors exchanges.

The appropriate time for the harvest of each spreading is judged as a function of the degree of degradation of the straw covering each spreading (appearance, change of color, etc.). This relies on the general knowledge of the person skilled in the art and on his or her common sense.

Step A4

6 Harvest:

At the end of the foregoing periods, the ENTIRETY of the degraded straw and of the degraded plants and vegetable matters together with their degradation products, including the fungi, is harvested, thus forming as many “vegetable complexes” CV as spreading piles, as represented by CV1 for the first fraction and CV2 for the second fraction.

The harvest takes place preferably one time per year, which obviously is not limitative.

The person skilled in the art will understand without difficulty that, since the degradation times of the first and second fractions are different, it will be advisable to prepare more or less numerous spreadings and distributed over time and according to whether the harvest will take place more or less rapidly.

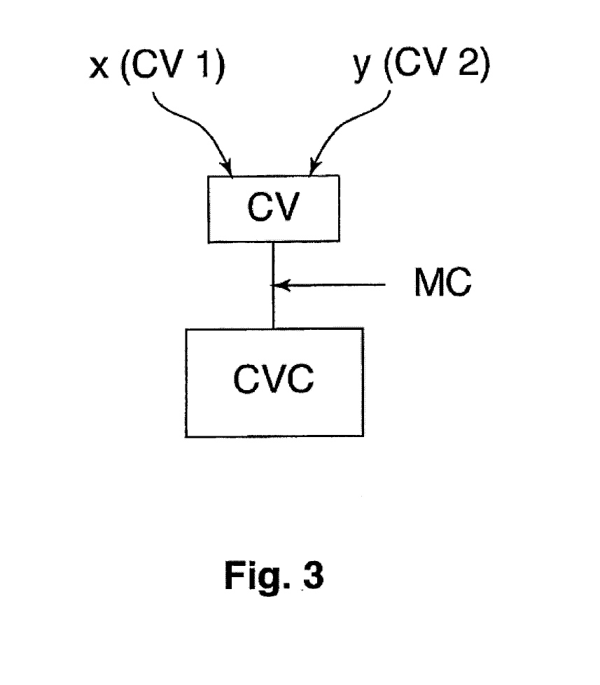

These harvests of vegetable complexes, i.e. xCV1, yCV2 (already “activated” by virtue of “doped” degradation) (as many CVi as “straw-covered spreadings”) are grouped and intermixed to form the “vegetable complex” CV (total) (which is therefore very activated and very complex (FIG. 3).

Each vegetable complex CV i contains residues and degradation products of the starting plant/vegetable matter and of the straw P used for cover, as well as the microfauna and microflora which, either were already present and have developed very extensively, or evolved in high proportion, especially with extraordinary multiplication of all kinds of fungi and spores, a factor considered by the Applicant to be one of the keys to the surprising properties of the intermediate products (total CV and carbonated CV, see hereinafter) and final products (doped vegetable complexes or (depending on the preparation option, see hereinafter) “superdoped” vegetable complexes).

Step B

Preparation of Highly Active “Carbonated Vegetable Complexes” (CVC)

(FIG. 3)

The (activated) total vegetable complex CV prepared in step B is
dried to a concentration of dry matters of approximately 60 to 80% by weight, and is then
mixed with a “carbonate matter”.

This “carbonate matter” may be defined as any matter containing a high or very high proportion of carbonate, most preferably calcium carbonate, in a form having coarse to very coarse particle sizes: the GCCs or ground natural calcium carbonates, of any type well known in the field of “mineral fillers”, for example marble, calcite, aragonite, etc., will therefore be preferred.

The particle size will be in the range of 100 to 300 microns, by way of indication, or even coarser.

The invention absolutely does not seek a homogeneous particle size, and to the contrary the particle size may be very heterogeneous. In addition, it will be inevitable that the carbonate material could comprise a small percentage of fines or ultrafines (inevitable in grinding) and/or of very coarse particles.

It will be noted that a heterogeneous or even very heterogeneous particle size will be preferred, so that the ambient atmosphere (see “Option 2” process hereinafter) is able to pass easily through the carbonate layer.

Lists of such carbonates are available to the person skilled in the art in the patents and documents dealing with grinding and additives for grinding of carbonates, with mineral fillers and additives for plastics, paints, paper, coating formulations for paper, etc., and the particle-size curves, d50, etc. are well known therefrom.

The preferred effective proportion of “carbonate matter” and especially of calcium carbonate is on the order of 70 to 95% by weight of dry carbonate matter (in general, the fraction of dry matters in a carbonate is on the order of 98% by weight), preferably 80 to 90%, the remainder up to 100% being the total vegetable complex containing 60-80% by weight of dry matters, i.e. from 5 to 30%, preferably from 10 to 20% of raw vegetable complex.

Process for Preparation of Doped or Superdoped Vegetable Complexes

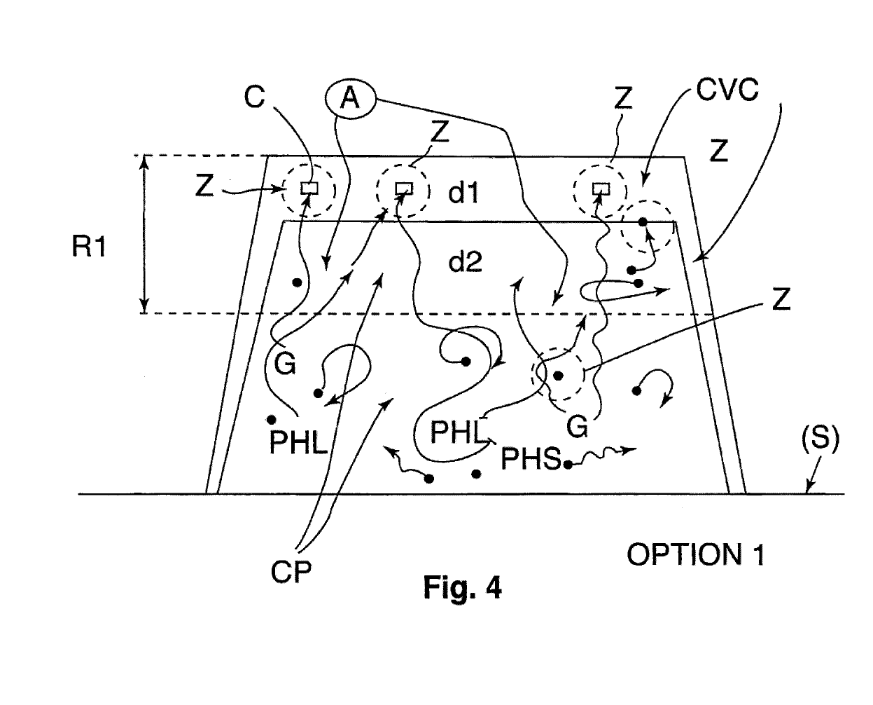

(FIGS. 4, 5, 6)

Option 1

Doped Vegetable Complex (CVD)

Step 1A

A compost is prepared with a very high proportion of straw or equivalent vegetable matter, most preferably of straw, and of an organic matter.

Depending on the mode that is most effective at this time and the most practical on the site, a compost of straw and horse dung will preferably be used.

Preferably this compost contains 90% by weight of straw and 10% by weight of horse dung.

Also preferably, this compost contains 20 to 40% of dry matters by weight, the balance being water.

It will be very clearly preferred to dispose a covering compost CVC on a different compost CP, for example (non-limitatively)
very preferably, a vegetable compost on a manure from cattle or horse breeding (equids)
or a vegetable compost on a manure from breeding of other animals.

It is pointed out that the proportion of animal matters is ultimately always smaller than 1% by weight gross in the matters provided to the microorganisms for development thereof.

Step 1B

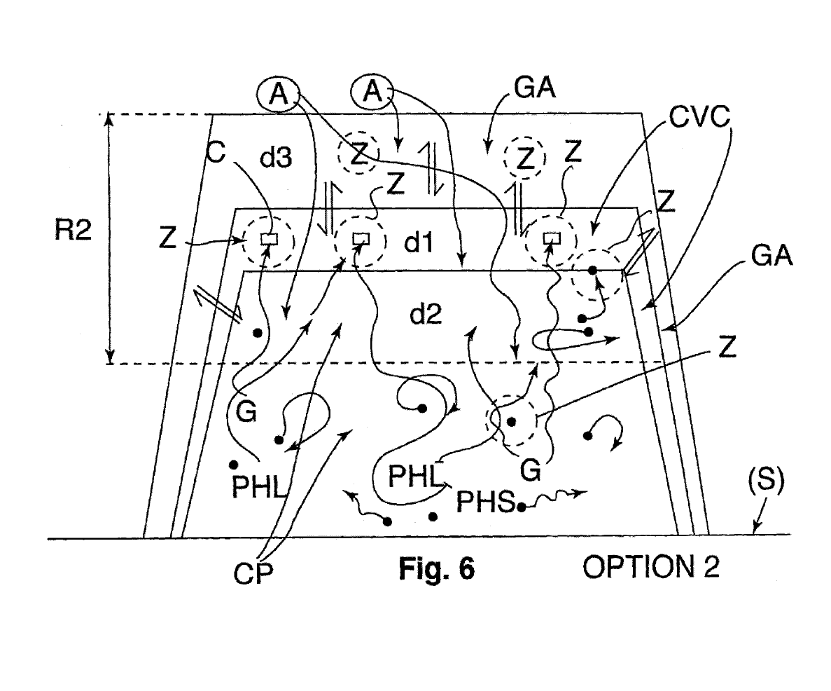

This compost is left to ferment under aerobic conditions for 3 to 6 days, thus producing the fermented or fermenting compost CP in FIGS. 4 and 6.

Step 1C

This compost CP is disposed on the soil in the form of piles of generally trapezoidal cross section, and it is covered on all its faces, especially its top face, with a layer of carbonated vegetable complex CVC obtained hereinabove.

The height of the pile of compost CP is on the order of 1.70 to 1.90 m.

On the top face of this pile of compost CP, the layer of carbonated vegetable complex CVC is on the order of 15 to 35 cm, preferably 20 to 30 cm, and from 5 to 10 cm on the side faces.

Step 1D

The compost CP is allowed to continue its fermentation under the cover of the covering layer CVC and in synergy therewith, until release of gas is observed at the top of the assembly. An apparatus for measuring the released gases is placed on top of the pile of compost CP to determine this state of maturation, although even without this apparatus such a determination is within the scope of the technical knowledge of any person skilled in the art and of his or her common sense.

Step 1E Harvest

When the person skilled in the art estimates that the complex is ready for harvest, he or she undertakes harvesting of: (see FIG. 4 Option 1)
all of the top and side layers of carbonated vegetable complex CVC (complex volume d1) AND of a volume d2 of compost CP situated underneath the interface between CVC and CP.

For a height of the mass of compost CP equal to 60-80 cm and a height of the top layer of CVC equal to 15-30, preferably 20-30 cm, the height of the volume d2 of the mass of compost CP taken underneath the CP/CVC interface is on the order of 60 to 80 cm.

Of course, reactions and exchanges take place throughout the lower compost CP and therefore also below the volume d2 that will be recovered, but it is evident that these events decline progressively (according to a gradient effect) with increasing distance from the said CVC/CP interface. Thus the CVC (d1) and a volume d2 of compost CP are recovered, which is a compromise between the need to obtain the largest possible recovered volume d2, but not too large so as not to recover a compost d2 that is too slightly activated, because it is too far from the interface, which would degrade the total activity of the recovered complex d1+d2″.

This compromise can be easily achieved by the person skilled in the art, if necessary with the assistance of some routine tests, for example by performing some tests on small volumes, such as 100 or 200 liters, by measuring the temperature, for example by a temperature sensor placed at the interface, by establishing (by taking samples of the compost around the zone of the sensor) a relationship between temperature and bioactivity, or by any other simple and pragmatic method. Such methods are known or easily accessible to any person skilled in the art.

As regards the second possible test factor (with the temperature), i.e. the “bioactivity” of the sampled special compost, it is easily possible, for example, to measure and analyze, continuously or intermittently, the volumes of gas being released; it will be possible, for example, to follow the release of CO₂ or NH₃ and, just as for the temperature, to establish a simple relationship such as bioactivity=f(CO₂ or NH₃ concentration) or analogous relationship within the capability of any person skilled in the art.

There have been noted for the harvested product d1 (CVC)+d2 (CP) (or at least the Applicant explains the properties and contents obtained only by this reasonable hypothesis):
a reorganization of the vegetable and organic matters, especially those derived from the straw and from the dung or manure;
an intervention of the gases of the ambient atmosphere (A), such as especially nitrogen, CO₂, O₂, H₂, which participate in the interphase exchanges in layers d1 and d2;
the effective intervention of the carbonate, which participates extensively in the fixation of the ammonia, CO₂, H₂ gases generated by the interphase exchanges or introduced via the ambient atmosphere (A);
an extremely high concentration of fungi or spores, a part of which has favored the vigorous development of humic acid nodules.

As pointed out hereinabove, the Applicant believes that the surprising properties obtained result from
the very vigorous proliferation of fungi, molds and similar species and from mycorrhizae,
and it also seems that the extremely extensive fixation of gases such as ammonia and CO₂ is essential for forming a “carbon and nitrogen sink”.

There basically are noted:
humic acids and their nodules,
fulvic acids,
aerobic microorganisms, especially fungi, actinomycetes and aerobic bacteria, spores, molds, etc.

In addition, the humic acid nodules and the humic acids, formed in extremely large proportions by virtue of the fungi, spores, etc., contribute to an essential large extent to the properties.

By “extremely extensive”, it is pointed out here that the concentrations are very largely greater than those that could be expected in view of the knowledge from the prior art and from the practices of the person skilled in the art, which is obviously surprising since it involves fundamental products that have been manipulated for numerous decades.

Without wishing to be bound by any theory, the Applicant suggests that the fact of “covering” a compost CP with a layer of activated and carbonated vegetable complex CVC will
a) largely block or “fix” the gaseous releases from the covered compost CP, which gaseous releases (CO₂, NH₃, H₂, etc.) will therefore be forced to “percolate” slowly through the elements of fermentation/decomposition of the compost CP; this percolation has to generate numerous exchanges between phases and between ingredients, including gas phase><solid phase><liquid or pasty phase, which will be slow and therefore extremely effective. The carbonate will assist surprisingly in fixing these gases, both those of the atmosphere and those of the compost CP.
b) permit bacteria to develop to the maximum and therefore to produce maximum doping effects (including action on fermentation, more active generation of gas, etc.);
c) permit additional exchanges with the carbonate matter;
d) give the atmospheric gases (A) time to intervene in the CVC layer and d1 layer of CP and to interact there;

It is for blocking in particular the escape of gases that it is necessary to cover the surface of the compost CP to the maximum and if possible in its entirety.

The “carbonate matter” C used will intervene, also according to a theory of the Applicant, not only via the known properties of a carbonate (which is a basic product) but above all also by the fact of forcing the moving liquid and gaseous substances to come into contact with coarse particles:
offering a large additional contact surface, which is favorable to the intensification of exchanges,
offering possibilities of adsorption/absorption of certain components of the composts at the surface of the carbonate particles,
and creating obstacles and tortuous paths for percolation and movements of phases, thus again offering opportunities for exchanges while favoring intermixing of the phases.

Thus, according to the invention, and contrary to the aerobic evolution of a farm compost, for example, loss of carbon or nitrogen or other useful elements does not occur, because there is no loss of fermentation gas: instead, the invention uses these gases (in combination with the bacterial actions) to participate in doping and enrichment with nutrients and active elements of the compost CP, especially in its recoverable part d2, as well as of the CVC itself.

An attempt has been made to schematize these phenomena in the attached FIG. 4, obviously very condensed.

PHL represents the liquid phases kept in motion by natural or thermal convection (heat of fermentation).

PHS (black dots) represents less mobile solid phases.

G represents the cited gases (O₂, NH₃, H₂, CO₂), which will percolate very slowly through the masses CP and d2, held back by the cover CVC and by the particles of carbonate C (white squares), which possess their inherent properties of adsorption/absorption, etc.

Z and the dashed circles each represent some of the zones of interphase exchanges, which in fact are present everywhere in the masses, between multiple combinations of phases, impossible to represent.

It is seen that the atmospheric gases A are able to penetrate into the layer CVD d1 and at least d2 and intervene: as products according to the invention, among other surprising properties, “fix” these gases, especially N2 and CO2 (carbon); this contribution is important.

The arrow lines schematically represent the possible movements, percolation, convection, Brownian movements, etc.

Step 1F Transfer onto the Screen or Sieve T

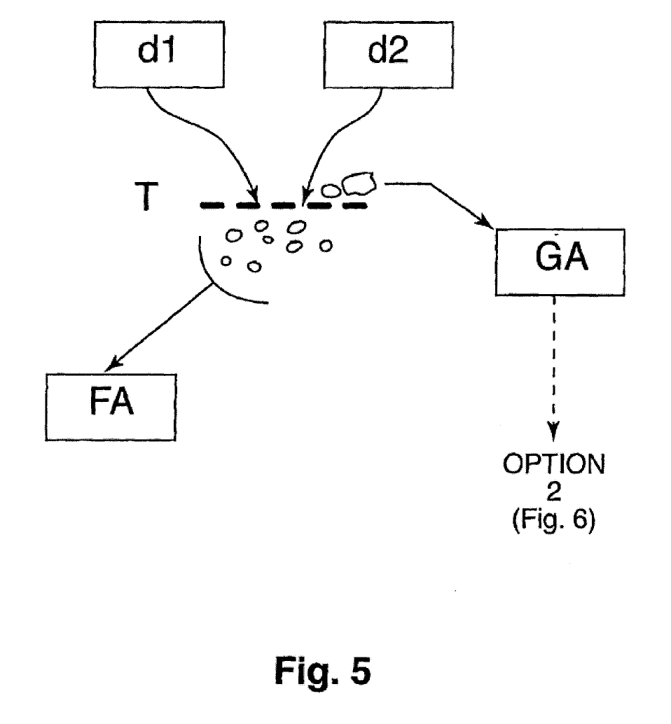

(FIG. 5)

The harvested “doped vegetable complex” CVD (in fact, in the preferred embodiment, it is a complex of degraded vegetable matters doped by a fermented compost of straw and horse dung, therefore containing a fraction of organic matters), in other words the volume d1 (CVD) and d2 (compost CP) is transferred onto a screen or sieve T, which retains the particles having sizes between approximately 1 and 10 mm, which constitutes a first product comprising “activated coarse particles” or “GA”, and allows the particles having sizes smaller than approximately 1 mm to pass, referred to here as “activated fine” particles or “FA”.

The finest particles are used as is or diluted in the applications considered. See “Dilution” section hereinafter.

As regards the activated coarse particles GA, which according to the Applicant's theory represent an important element of the invention, they will be used in the same process as hereinabove, but as an additional top layer, to produce a “superdoped vegetable complex” (containing, as hereinabove, a fraction of doping by a fraction of organic matter).

Option 2

Superdoped Vegetable Complex (CVSD)

The steps are strictly the same as in option 1 except that, after the compost CP has been covered by the layer of CVC (carbonated vegetable complex), the whole is covered by a third layer composed of activated coarse particles GA obtained as sieve rejects in option 1 hereinabove.

See FIG. 6.

The phenomena and interphase exchanges are the same, except that the complex CVD is activated “in a sandwich” both by the covered compost CP and by the layer of activated coarse particles GA (which allows the atmospheric gases A to penetrate by virtue of its natural asperity).

One of the advantages of the layer GA, in addition to that of imposing yet more slowing of the percolation and therefore even better fixation of the gases and production of humic acids, is to contribute humic acid nodules and a high concentration of fungi to the reactive system. Hence the notion of activation “in a sandwich”.

The effects and the obtained products will be essentially the same as in option 1, except that the “doping” will be more pronounced, by approximately 10 to 20%, and that the particles GA, otherwise difficult to employ on site, will have been meaningfully reused.

The exchange phenomena are represented in the attached FIG. 6. They are the same as those explained in connection with the attached FIG. 4 (option 1), except that the layer GA permits additional exchanges (it is recalled that CP and GA are strongly activated at the same time), represented schematically by the double arrows.

As in option 1, d1 of CVD and d2 of CP are recovered, plus the layer GA (generally 40-50-60 cm at the top), the ratio between everything that is harvested (d1+d2+GA) and the unused remainder of the CP then being on the order of 50/50 by volume.

After screening, a superdoped vegetable complex is obtained that can be used as is or diluted (see “Dilution” section hereinafter).

In fact, one of the achievements of the Applicant is having thought about blocking or slowing the losses of gas during fermentation, specifically by combining three composts, one of which was undergoing aerobic fermentation and is then covered by a carbonated compost and by a coarse compost.

The invention therefore achieves a combination of blocking of gases, including hydrogen, and of fixation of other gases, including CO₂, in the mass: hence the absence of losses of carbon, nitrogen and other useful elements, i.e. the creation of a “carbon and nitrogen sink”.

Novel Industrial Products

The two process options therefore result in two “novel industrial products” CVD and CVSD, by virtue of their “bioactivity” and their composition, the novelty of which (see hereinafter) can be established by “markers” such as:
the fact that the gases NH₃, CO₂, also H₂ “blocked” in this way may then be fixed, probably by action of hydrogen, which is itself also blocked. Thus the Applicant has noted that, according to the invention, 1 metric ton of composted manure 2 according to the invention yielded 840 kg of residual product, while a normal aerobic farm manure, for example, allowed only 600 kg to remain after the same time period, 6 months. The extra concentration achieved by the invention is therefore measured, as is the conservation of useful elements (carbon, nitrogen), which in a normal manure disappear (often in the toxic form of CO₂ or ammonia).
The concentration of humic acid nodules and humic acids (from 200 to 600% (especially 300-500%)) greater than the concentration expected by the person skilled in the art without using the “covering” process of the invention.
The concentration and the diversity of microorganisms (bacteria, fungi, molds, etc.), which in turn will act to produce humic acids.
An exceptional concentration of mycorrhizae, which are fungi that become fixed on the roots and are capable of seeking water at a depth 5 to 10 times greater than the roots, to the benefit of the plants.

Potentially, this product may also be characterized and differentiated from the traditional composts by virtue of an intermediate “marker”, which is the hemicellulose ratio in the complex CVD or CVSD; compared with a traditional compost, this ratio is effectively approximately double in a product according to the invention and ready for use and having been recovered at the end of 6 to 8 months of interactions.

Dilution: Complex CVD or CVSD According to the Invention and its Different Presentation Variants:

“Pure” CVD or CVSD: the pure products are extremely active and concentrated in active matters, including the humic acid nodules, with fixation of very high (very surprising) doses of carbon and nitrogen.

This pure product may be used as is, i.e. without dilution. Nevertheless, it is obviously very concentrated, and the use of such a concentrated product may cause practical problems (unsuitable equipment, risk of overdosing, etc.).

Dilution with a “Neutral” or “Inert” Support

The pure complex CVD or CVSD (either one) according to the invention may be diluted with one or more inert or neutral solid supports, such as carbonate or silica sand or other products such as pozzolans, etc. The dilution may be applied in almost any proportion, which will be at the discretion of the final user, especially in a ratio of pure CVD or CVSD to sand (by gross weight) equal to 30/70, 20/80, or even 10/90, 5/95, 1/99, because the product is active and effective.

Dilution with a Support of “Organic Matters” Type (or Containing Such Organic Matters)

The dilution may be applied with all kinds of products containing a percentage of known organic matters, such as rapeseed cake, olive pulp, cocoa beans, etc.

The invention encompasses these novel industrial products, whether pure or diluted, and in general the novel industrial products characterized in that they consist of or comprise at least one CVD or CVSD or pure and/or dilute mixtures thereof.

The product may be used alone in a proportion of 200 to 500 g per metric ton of manure. In a second case, it may be mixed with a mineral matter (product that goes into the manure with 5 to 20% of the core material), and this product will be spread in the dose of 1 to 5 kg per metric ton of manure.

The product to be spread in the fields may be used in a proportion of 20 to 40 kg per hectare when it is pure. Since it is very difficult to distribute it homogeneously in such a dose, it is expanded with mineral and organic matters corresponding to 5 to 10% of the core material and is spread on the soils in a proportion of 200 to 600 kg per hectare.

Surprisingly, there has now been discovered a totally unexpected application of these products for production of biogas containing methane or of methane itself, with a yield ranging from “improved” (when the starting organic matters are not very rich) to “extremely increased” when the starting organic matters are normal to excellent.

Methanation/Methane Production/Production of Biogas Containing Methane

Traditional “methanizers” are known: fermentation is brought about in open or closed fermenters, for example 3000 m³ of diverse organic wastes such as: muds from treatment plants, industrial residues containing organic components such as greases, food wastes, slurry, diverse manures, obviously with a maximum concentration of organic matters, which are supplemented each day in this volume example by approximately 60 metric tons of the same types of organic wastes, and in this way a biogas containing in particular, among other components, methane CH₄ (between 3 and 5 metric tons), is produced each day.

If, according to the invention, merely 600 kg of pure CVD or CVSD according to the invention, diluted with sand in the proportions indicated hereinabove, is added to and mixed with this daily supplement of 60 metric tons of organic wastes, the yield of CH₄ is multiplied by 120 to 200-220-350%, depending on the starting organic wastes. Even a “small” gain of 20% represents a certain advance. If the operation is taking place with rich organic media such as treatment muds, the highest values are attained. It is also noted that the H2S odor is not as strong.

The invention also extends to biogas containing methane and to methane itself, characterized in that it was obtained by the process according to the invention.

The invention makes it possible to increase the production of biogas and the proportion of methane in such biogas, which is equivalent to increasing the production of methane by 1.1 to 6 times more and generally by 1.3 to 3 times more and to reducing the proportion of CO₂ in the biogas by 10 to 90% and generally by 40 to 60%.

In addition, reactor bottoms known as “digestate” remain in the methanation reactor. This product has the form of semi-liquid mud.

When the product according to the invention has been used for the production of methane, this digestate exhibits extremely advantageous properties.

It contains approximately two or three times as much humic acids (especially 140 to 200% more) and, in addition, which is a very important factor, the organic matters are “organized” into assimilable products, while the nitrogen is much better fixed, in particular by the humic acids.

According to a non-limitative theory, the Applicant considers that the humic acids are able to flocculate with the clay of the soil during spreading of the digestate, these flocculates becoming intercalated between the platelets of clay, which will contribute to aerating the soil and to permitting the roots to become inserted into the clay until they make contact with the acids, where precisely the nutritive nitrogen is fixed.

For the “methanation” application, it will be advisable very preferably to use a carbonate sand in a proportion of 35 to 60% by weight expressed as CaO, preferably from 45 to 54% by weight relative to the pure special compost product of the invention.

This gas may be used in all of its known applications.

The invention further relates to a novel industrial product, characterized in that it consists of the recovered digestate, which is characterized in that it contains approximately two or three times as much humic acids (especially from 140 to 200% more) compared with the traditional digestates without the use of CVD or CVSD.

The invention further relates to the applications of the methane obtained by the process according to the invention in all of its known applications.

1. A method for preparing doped (CVD) or superdoped (CVSD) vegetable complexes, said method comprising:
a) covering a spreading of a plant complex with untreated straw, wherein the plant complex (CPL) is obtained from a plant or particular vegetable matter, and
a1) degrading said covered plant complex either outside the soil for 2 to 8 months or half-buried in the soil for 18 to 24 months,
a2) harvesting of said degradation products from a1) thereby forming a vegetable complex (CV);
b) drying the CV to a concentration of approximately 60-80% of dry matter;
c) mixing the dried CV with a carbonate matter in proportions of 70 to 95% by weight of carbonate matter, thus forming a carbonated vegetable complex (CVC);
d) fermenting aerobically, in parallel and separate from the CV, a vegetable compost comprising approximately 5-15% of organic matters, for 3-6 days thereby producing a fermented or fermenting compost (CP);
e) covering a layer of CP with CVC and optionally a top layer of an activated coarse matter (GA);
f) harvesting an upper part (d2) of the compost CP situated underneath the interface with the layer of CVC when gaseous release is no longer observed at the top of the pile thereby producing crude CVD or, if the layer of GA was present, thereby producing crude CVSD;
f1) transferring the crude CVD or crude CVSD onto a 1-mm sieve, and collecting the fine particles (FA) which pass through the sieve and the activated coarse matter (GA) which does not pass through the sieve, wherein the FA is pure CVD or pure CVSD.

2. (canceled)

3. (canceled)

4. The CVD or the CVSD obtained by the process according to claim 1.

5. Novel industrial products comprising at least one CVD, CVSD or combination thereof from claim 1.

6. (canceled)

7. A method of methanation, production of methane or production of biogas comprising methane, said method comprising introducing at least one CVD or CVSD according to claim 4 into a methanizer.

8. The method according to claim 7, wherein for a 3000 m³ methanizer using organic wastes, and a daily supplement of 60 metric tons of these organic wastes, 600 kg of pure CVD or CVSD, which has been diluted with sand or a carbonate sand, is added to and mixed with the daily supplement, in a proportion of 35 to 60% by weight expressed as CaO, relative to the pure product CVD or CVSD.

9. The method according to claim 7, wherein a reactor bottom known as digestate is recovered.

10. A novel industrial product which comprises the digestate recovered according to claim 9 wherein the humic acid content is approximately two or three times higher than compared to a traditional digestate.

11. Biogas comprising methane, produced by the method according to claim 7.

12. (canceled)
